(19) United States
(12) Patent Application Publication
(10) Pub. No.: US 2026/0192121 A1
(43) Pub. Date: Jul. 9, 2026

(54) INTEGRATED BIOELECTRIC THERAPY SYSTEM WITH PRACTITIONER-ASSISTED CONDUCTION INTERFACE, ELECTROMAGNETIC CONDITIONING, AND SENSOR-BASED ASSESSMENT

(71) Applicant: Daniel O. Lopez, San Antonio, TX (US)

(72) Inventor: Daniel O. Lopez, San Antonio, TX (US)

(21) Appl. No.: 19/553,998

(22) Filed: Mar. 2, 2026

Related U.S. Application Data
(63) Continuation of application No. 18/395,182, filed on Dec. 22, 2023.

(51) Int. Cl. A61N 2/00

(52) U.S. Cl. CPC A61N 2/008

(57) ABSTRACT

A bioelectric therapy system includes a pulsed electromagnetic field module for tissue conditioning and, in some embodiments, a grounding interface configured to electrically couple a subject to a ground reference; a bioelectric stimulation module configured to deliver a regulated, current-limited stimulation signal in a selected low-voltage range via a practitioner-assisted conduction path; and an analyzer configured to acquire measurable biophysical signals to generate assessment data representing physiological parameters. A control unit coordinates a protocol including conditioning, grounding, and stimulation, modifies at least one stimulation parameter based on assessment data or a monitored electrical characteristic, and generates a health assessment report (e.g., a color-coded assessment report) for longitudinal tracking.

### CROSS-REFERENCE TO RELATED APPLICATIONS

Not applicable.

### STATEMENT REGARDING FEDERALLY SPONSORED RESEARCH OR DEVELOPMENT

Not applicable.

### FIELD

The present disclosure relates generally to bioelectromagnetic therapy systems and methods. More particularly, the disclosure relates to integrated therapy platforms that coordinate (i) pulsed electromagnetic field (PEMF) conditioning, (ii) bioelectric stimulation delivered via a practitioner-assisted conduction interface, and (iii) sensor-based acquisition of measurable biophysical signals to generate assessment data and reports, including protocols aligned to defined anatomical zones.

### BACKGROUND

Chronic pain, inflammation, and functional impairment represent significant healthcare challenges. Conventional interventions may include pharmacologic regimens and invasive procedures that can be associated with adverse effects, contraindications, dependency risks, or limited long-term efficacy.

Complementary modalities such as acupuncture, pulsed electromagnetic field therapy, and low-voltage bioelectric stimulation have been used to support pain management and wellness. In practice, electrostimulation performance can depend on establishing effective grounding and delivering stimulation at a selected voltage level suitable for a given subject and protocol. However, these approaches are frequently implemented as isolated techniques, with limited standardization of sequencing, limited integration of voltage regulation, safety monitoring, and contact verification, and limited objective longitudinal tracking of session-to-session response.

In addition, conventional electrotherapy approaches often rely on electrode placement directly at treatment points, which can be inconsistent across operators and may not provide a standardized mechanism for distributing stimulation across multiple anatomical regions in a structured sequence.

Accordingly, a need exists for integrated therapy systems and methods that (i) condition tissue using controlled PEMF exposure, (ii) deliver bioelectric stimulation through a practitioner-assisted conduction interface with safety gating and monitoring, and (iii) generate assessment reports to support repeatable protocols and objective tracking over time.

### BRIEF SUMMARY

The present disclosure provides systems, methods, and apparatus for bioelectric therapy integrating electromagnetic conditioning, practitioner-assisted stimulation, and sensor-based assessment and reporting. Embodiments may be implemented as systems, methods, apparatus, and/or computer-readable media.

In some embodiments, a bioelectric therapy system comprises: (i) a PEMF module configured to generate controlled electromagnetic pulses to condition biological tissue; (ii) a bioelectric stimulation module configured to deliver a current-limited stimulation signal through a practitioner-assisted conduction path; (iii) an analyzer configured to acquire one or more measurable biophysical signals associated with a subject and generate assessment data representing physiological parameters; and (iv) a control unit configured to coordinate a therapeutic protocol and generate a health assessment report based on the assessment data. In some embodiments, the system further includes a grounding interface configured to electrically couple the subject to a ground reference during at least one of PEMF conditioning and stimulation, and the stimulation signal is regulated to a selected low-voltage range suitable for the protocol. In some embodiments, the health assessment report is presented as a color-coded assessment report in which parameter classifications are mapped to colors (e.g., green=normal, blue=mildly abnormal, yellow=moderately abnormal, and red=severely abnormal and/or abnormal depending on testing category) to visually convey parameter classifications and/or severity ranges.

In some embodiments, the practitioner-assisted conduction path includes a subject electrode pad electrically coupled to a first electrode terminal and a practitioner return pad electrically coupled to a second electrode terminal, such that electrical current flows to targeted anatomical regions through a practitioner's hands when the practitioner contacts the practitioner return pad and touches the subject. In some embodiments, the system includes contact detection and safety interlocks to inhibit or terminate stimulation when contact is lost or when monitored electrical characteristics exceed thresholds.

In some embodiments, PEMF conditioning is selected to influence one or more measurable biophysical signals prior to stimulation to support repeatability. In some embodiments, the control unit modifies one or more stimulation parameters based on at least one of (i) the assessment data or (ii) a monitored conductive condition associated with the practitioner-assisted conduction path.

These and other features, functions, and advantages will be apparent from the following description and accompanying drawings.

### BRIEF DESCRIPTION OF THE DRAWINGS

The accompanying drawings, which form part of this disclosure, illustrate various embodiments. The drawings are not necessarily to scale. Like reference numerals may denote like elements.

FIG. 1 is a schematic diagram illustrating an example bioelectric therapy system 100 including a subject 10 and a practitioner 20, and further illustrating a pulsed electromagnetic field (PEMF) module 110, a bioelectric stimulation module, a health assessment module 140, and a user interface 150. FIG. 1 also illustrates an example practitioner-assisted conduction interface including a subject electrode pad 322 and a practitioner return pad 332 for delivering a current-limited stimulation signal when the practitioner 20 contacts the return pad 332 and touches the subject 10, according to some embodiments.

FIG. 2 is a schematic diagram illustrating an example electromagnetic conditioning arrangement including a field driver 220, coil assemblies 210, and a subject positioning apparatus 230 that delivers a pulsed electromagnetic field during a conditioning phase 420 according to some embodiments.

FIG. 3 is a schematic diagram illustrating an example practitioner-assisted conduction configuration including a stimulation circuit 310, one or more subject electrode pads 322, one or more practitioner return pads 332, a subject 10, and a practitioner 20 according to some embodiments.

FIG. 4 is a flowchart depicting an example bioelectric therapy method 400 including intake and baseline assessment 410, PEMF conditioning and grounding 420, optional topical preparation 430, setup and contact verification 435, multi-zone bioelectric stimulation 440, and session completion and report generation 450 (including, in some embodiments, post-stimulation assessment data acquisition) according to some embodiments.

FIG. 5 is a block diagram illustrating an example analyzer pipeline including sensor(s) 510, signal processing 520, analysis logic 530, a parameter library/reference database 540, and an assessment data output 550 according to some embodiments.

FIG. 6 is an example color-coded health assessment report 600 showing baseline and follow-up values with example classification outputs (e.g., severity class assignments), wherein green indicates normal, blue indicates mildly abnormal, yellow indicates moderately abnormal, and red indicates severely abnormal and/or abnormal depending on testing category, according to some embodiments. For purposes of black-and-white reproduction in patent drawings, the color categories may be depicted using different grayscale fills, hatch patterns, symbols, and/or other graphical indicators.

FIG. 7 is a flow diagram illustrating an example five-zone stimulation sequence 700 including zones 810, 820, 830, 840, and 850 according to some embodiments.

FIG. 8 is a diagram illustrating example anatomical zones corresponding to the five-zone stimulation sequence 700 according to some embodiments.

FIG. 9 is a flowchart depicting another example bioelectric therapy method 400, including optional decision loops and/or repeated steps, according to some embodiments.

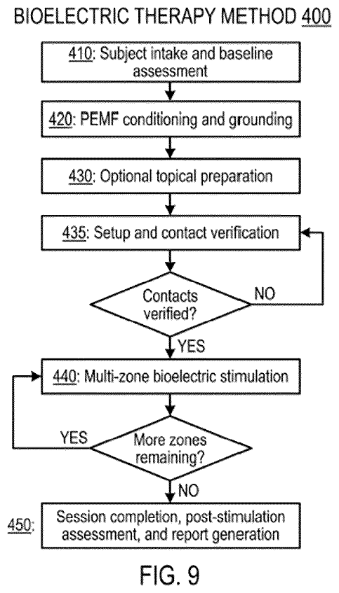

FIG. 10 is a block diagram illustrating an example safety and monitoring arrangement 1000 including an isolated power supply 350, controller/timing 140, waveform generator 360, current limiter 370, contact detection 380, an impedance monitor 390 (also referred to herein as a conduction monitor), a subject electrode pad 322, and a practitioner return pad 332 according to some embodiments.

### DETAILED DESCRIPTION

The following detailed description is provided for illustration and is not intended to be limiting. Various modifications, substitutions, and equivalents will be apparent to those of ordinary skill in the art and are intended to fall within the scope of the appended claims.

As used herein, the terms “comprising,” “including,” and “having” are inclusive and mean “including without limitation.” Unless otherwise indicated, the terms “a” and “an” are intended to include one or more.

### 0. Definitions and Design Considerations

As used herein, “low-voltage” refers to an output voltage at stimulation terminals that is limited to a level suitable for non-invasive contact. In some embodiments, the stimulation output is limited to less than approximately 30 volts peak. In certain embodiments, the stimulation output is limited to less than approximately 20 volts peak. In some embodiments, the control unit sets a voltage amplitude setpoint (or range) and the stimulation circuit regulates and/or verifies delivered voltage to support repeatable protocol execution.

As used herein, “current-limited” refers to output current limited by hardware (e.g., a current limiting circuit and/or series resistance) such that, under normal operation, output current remains below a selected maximum current. In some embodiments, the maximum output current is less than about 10 milliamps. In some embodiments, the maximum output current is less than about 5 milliamps.

As used herein, “assessment data” refers to measured and/or computed data derived from one or more measurable biophysical signals and/or resonance characteristics (e.g., resonance-related signals, skin conductance, pulse-related signals) acquired using sensors. The term “assessment” is not intended to require a medical diagnosis; rather, it refers to generation of parameter values and classifications for tracking and protocol optimization.

In some embodiments, the system includes safety features such as isolation (e.g., an isolated power supply), current limiting, contact detection, time limits, and/or automatic shutoff when contact is lost or when monitored electrical characteristics exceed thresholds. In some embodiments, the system includes a grounding interface configured to couple the subject and/or practitioner to a ground reference (e.g., earth ground or chassis ground) to support stable operation and repeatable measurements.

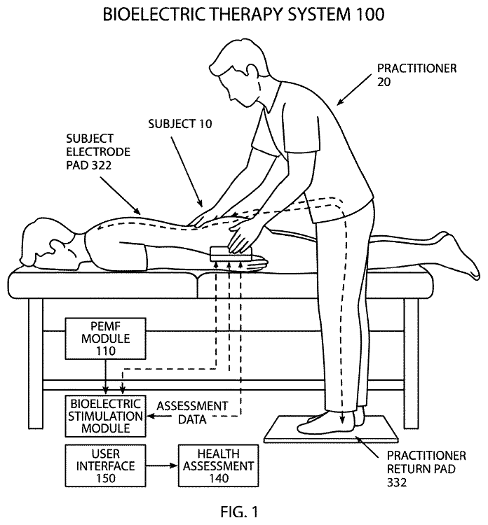

### 1. System Overview (fig. 1)

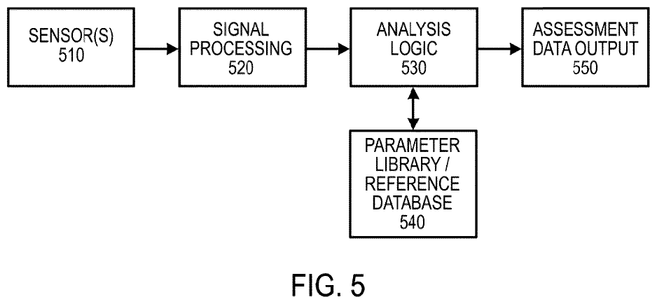

Referring to FIG. 1, a bioelectric therapy system 100 can be configured to deliver a therapeutic protocol to a subject 10 with assistance from a practitioner 20. In some embodiments, the system 100 includes a PEMF module 110, a bioelectric stimulation module, and a health assessment module 140 (also referred to herein as a controller and/or a control unit). In the illustrated example, a subject electrode pad 322 is coupled to the subject 10 and a practitioner return pad 332 is positioned for contact by the practitioner 20, such that a practitioner-assisted conduction path is formed when the practitioner contacts the return pad 332 and touches the subject 10. In some embodiments, the system 100 further includes a user interface 150 for session control and/or report presentation. In some embodiments, an analyzer (e.g., as described with respect to FIG. 5) acquires one or more measurable biophysical signals and generates assessment data for use by the health assessment module 140. In some embodiments, a data store stores session data, protocols, parameter libraries, and health assessment reports.

The control unit 140 can coordinate operating parameters (e.g., PEMF field settings, stimulation waveform, sequence timing, contact verification, safety interlocks, and report generation). The control unit 140 may be implemented using one or more processors and one or more non-transitory memory devices storing program instructions.

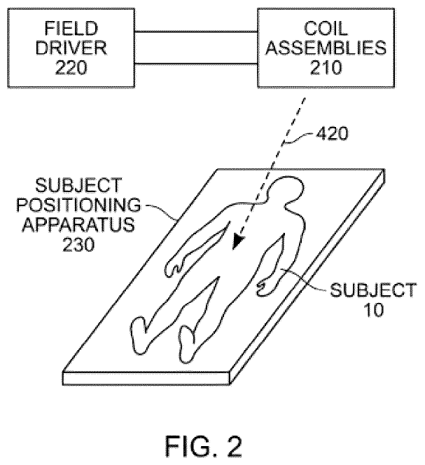

### 2. Pulsed Electromagnetic Field Module (fig. 2)

Referring to FIG. 2, the PEMF module 110 can include one or more electromagnetic coil assemblies 210 driven by a field driver 220. The PEMF module 110 can be integrated into a subject positioning apparatus 230 (e.g., a seat, mat, table, or applicator) to apply a pulsed electromagnetic field to a subject during a conditioning phase. In some embodiments, the subject positioning apparatus 230 includes or is coupled to a grounding interface (e.g., a conductive mat, strap, or grounding pad) configured to electrically couple the subject to a ground reference during the conditioning phase. The grounding interface may be used alone or concurrently with PEMF exposure.

In some embodiments, PEMF parameters include one or more of waveform, frequency, pulse width, duty cycle, and field strength. In some embodiments, the frequency range of the PEMF device is from about 10 Hz to about 30 Hz. In some embodiments, the PEMF module is operated for a predetermined duration and, in most embodiments, the PEMF application duration is approximately 8 minutes.

In some embodiments, the conditioning phase is selected to promote repeatable protocol execution and to prepare the subject for subsequent stimulation. In some embodiments, the control unit 140 selects or modifies a stimulation parameter (e.g., pulse amplitude, ramp rate, dwell time, and/or zone ordering) based on at least one of (i) baseline assessment data, (ii) post-conditioning assessment data, and/or (iii) stored session history.

### 3. Bioelectric Stimulation Module and Practitioner-Assisted Conduction (FIGS. 1, 3, and 10)

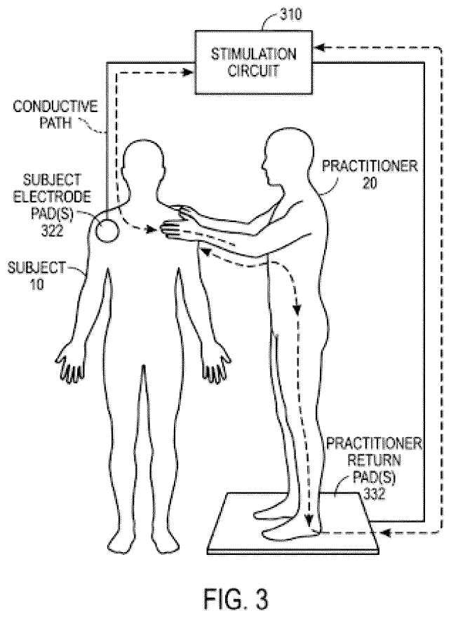

Referring to FIG. 3, a stimulation circuit 310 can be configured to generate a controlled, current-limited stimulation signal suitable for non-invasive application. The stimulation circuit 310 can be coupled to one or more subject electrode pad(s) 322 and one or more practitioner return pad(s) 332. In some embodiments, the stimulation circuit 310 includes voltage regulation and/or monitoring circuitry to maintain the stimulation signal within a selected low-voltage range and to verify delivered voltage and/or current during a session. In some embodiments, the stimulation circuit 310 is implemented by, or included within, a bioelectric stimulation module.

Referring to FIG. 1, in an example physical setup, the subject electrode pad 322 can be positioned at a subject contact region (e.g., the feet, buttocks, or other suitable location) to establish a stable subject contact. The practitioner return pad 332 can be positioned on a practitioner support surface (e.g., a floor pad). When the practitioner contacts the practitioner return pad 332 (e.g., by standing on it) and touches the subject with the practitioner's hands, a conductive path is formed through the practitioner such that stimulation current can be delivered to targeted anatomical regions through the practitioner's hands. In some embodiments, the subject is positioned prone (face-down) on a table during stimulation.

In some embodiments, the stimulation signal is a pulsed waveform (e.g., pulsed DC or biphasic pulses). By way of non-limiting example, stimulation frequency may be between about 0.5 Hz and about 500 Hz, a duty cycle may be between about 1% and about 50%, and a ramp-up interval may be between about 0.5 seconds and about 10 seconds. In some embodiments, the stimulation circuit 310 includes current limiting and isolation such that, under normal operation, output current remains below a selected maximum current.

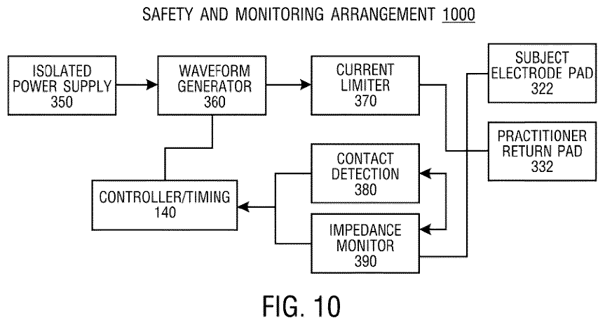

Referring to FIG. 10, the system can include contact detection circuitry and safety interlocks. For example, the system can inhibit or terminate stimulation unless (i) a subject contact is detected at the subject electrode pad 322, (ii) practitioner contact is detected at the practitioner return pad 332, and (iii) a conductive condition indicating practitioner hand contact is detected. In some embodiments, the system monitors a conductive condition and/or current stability and terminates stimulation when contact is lost or when monitored values fall outside thresholds.

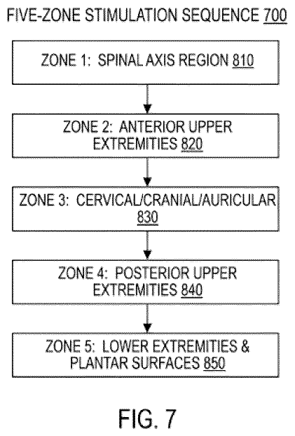

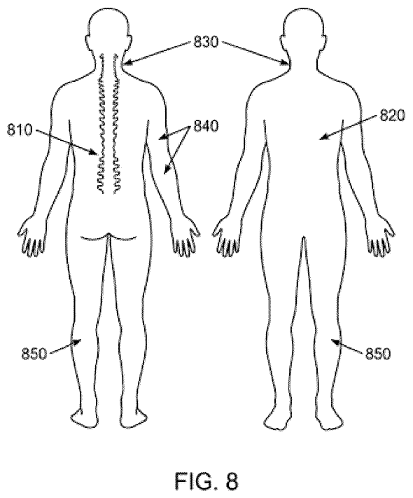

### 4. Stimulation Targeting, Zones, and Protocol Variations (FIGS. 7 and 8)

Bioelectric stimulation can be applied to one or more defined anatomical regions. In some embodiments, the zones correspond to anatomical landmarks and surface anatomy, and may be aligned, in a conceptual mapping, to meridian-based pathways described in Traditional Chinese Medicine. FIG. 8 illustrates an example set of anatomical zones 810-850, and FIG. 7 illustrates an example five-zone stimulation sequence 700.

In some embodiments, a five-zone sequence includes: (i) a spinal axis zone (including an occipital-to-sacral pathway); (ii) an anterior upper extremities zone; (iii) a cervical/cranial/auricular zone; (iv) a posterior upper extremities zone; and (v) a lower extremities and plantar surfaces zone.

In some embodiments, the system supports alternative zone partitions (e.g., 3 zones, 5 zones, 7 zones, or 9 zones) and alternative orderings. By way of example, ordering may be (i) fixed, (ii) symptom-driven, or (iii) analyzer-driven. In some embodiments, a dwell time per zone is between about 15 seconds and about 10 minutes, with optional stop conditions based on reaching a target window and/or analyzer-derived criteria.

### 5. Analyzer and Signal Acquisition (FIG. 5)

Referring to FIG. 5, the analyzer can include one or more sensors 510, signal processing circuitry 520, and analysis logic 530. In some embodiments, the analyzer acquires one or more measurable biophysical signals and/or resonance characteristics from the subject. By way of example, the sensors 510 can include sensors configured to measure galvanic skin response (skin conductance), pulse-related signals, and/or other measurable biophysical signals.

In some embodiments, measured signals are processed to compute features and parameter values usable for classification and tracking. In some embodiments, signal processing includes filtering, windowing, spectral analysis, normalization, and thresholding. In some embodiments, analysis logic 530 identifies resonance-related patterns relative to a reference library and generates per-parameter classifications.

In some embodiments, analysis logic 530 compares measured values and/or computed features to a parameter library 540 and assigns each parameter to a classification category. For example, a parameter may be classified into categories corresponding to normal, mild deviation, moderate abnormality, and severe abnormality. In some embodiments, the analyzer outputs baseline and post-stimulation assessment data, and the control unit computes session-to-session deltas and trends.

In some embodiments, the analyzer is a resonance-based analyzer. As used herein, “resonance-based” refers to processing of measured electrical signals to identify patterns relative to a reference library and is not limited to any particular brand or proprietary device.

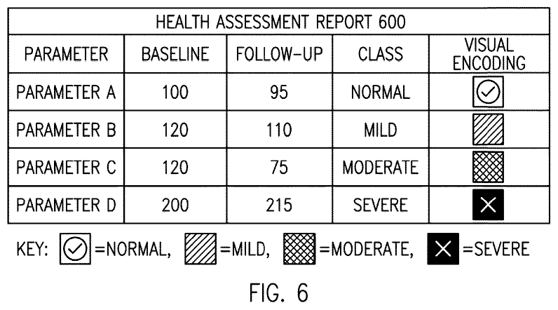

### 6. Health Assessment Report Generation and Longitudinal Tracking (FIG. 6)

Referring to FIG. 6, the control unit 140 can generate a health assessment report 600 based on assessment data from the analyzer. In some embodiments, the report 600 is presented as a color-coded assessment report utilizing color coding only, in which parameter classifications are mapped to colors to provide rapid visual interpretation. By way of example, green may indicate normal, blue may indicate mildly abnormal, yellow may indicate moderately abnormal, and red may indicate severely abnormal and/or abnormal depending on testing category. For purposes of black-and-white reproduction in patent drawings, FIG. 6 may depict the color categories using different grayscale fills, hatch patterns, symbols, and/or other graphical indicators; such indicators are used only to illustrate the underlying color categories and do not require that the delivered report incorporate shading, patterns, or symbols. The report 600 can present baseline and follow-up parameter values and corresponding classifications and can store session-to-session comparisons. In some embodiments, analyzer output is imported into a spreadsheet-based processing system (e.g., automatically transferred and/or manually entered) to compute classifications and render the report 600.

In some embodiments, the report 600 includes an aggregate index reflecting a ratio of parameters classified as normal to a total number of evaluated parameters. In some embodiments, the report includes baseline and follow-up measurements associated with a single session and/or trends across multiple sessions.

In some embodiments, the report 600 is stored in the data store with metadata including subject identifiers, date/time, protocol parameters, and device identifiers.

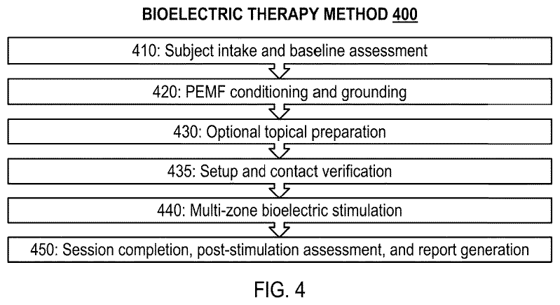

### 7. Method of Operation (FIG. 4)

Referring to FIG. 4, a method 400 can include: (i) subject intake and baseline assessment (step 410), including informed consent, collection of biometric data, and acquisition of baseline assessment data; (ii) PEMF exposure and/or grounding (step 420) for a predetermined duration; (iii) optional topical preparation (step 430), such as application of oils and/or creams to defined regions; (iv) stimulation setup and safety verification (step 435), including detection of subject electrode pad contact, practitioner return pad contact, and a conductive condition indicative of practitioner hand contact with the subject; (v) practitioner-assisted bioelectric stimulation across zones (step 440); and (vi) session completion and report generation (step 450), which can include acquiring post-stimulation assessment data and generating a health assessment report that compares baseline assessment data to post-stimulation assessment data.

In some embodiments, during step 440, the control unit ramps stimulation output only after confirming circuit closure, and ramps down or terminates stimulation when contact is lost. In some embodiments, the control unit adjusts one or more stimulation parameters based on monitored contact condition and/or analyzer output to maintain stability and protocol consistency.

### 8. Example Variations and Optional Features

In some embodiments, the system 100 includes closed-loop control that selects or adjusts stimulation parameters based on contact verification status and/or analyzer-derived trends. In some embodiments, the control unit includes software configured to recommend zone ordering, dwell times, and/or PEMF parameter sets based on stored session histories using a rule set and/or a look-up table mapping measured values to stored protocol parameter sets.

In some embodiments, a color-coded health assessment report is generated and delivered to a subject via electronic mail transfer. In some embodiments, the system is configured as a portable kit including a stimulation module, subject electrode pad(s), practitioner return pad(s), and a compact PEMF applicator.

### 9. Advantages

Embodiments can coordinate electromagnetic conditioning, practitioner-assisted stimulation with contact verification and safety gating, and standardized reporting within a repeatable protocol to support objective tracking and protocol optimization as a non-invasive adjunct for wellness and pain management.

While the disclosure has been described with respect to specific embodiments, modifications and variations may be made without departing from the scope of the disclosure as defined by the claims.

What is claimed is:

1. A bioelectric therapy system comprising:
a pulsed electromagnetic field module configured to generate controlled electromagnetic pulses for tissue conditioning;
a bioelectric stimulation module configured to deliver a current-limited stimulation signal via a practitioner-assisted conduction path;
an analyzer configured to acquire one or more measurable biophysical signals associated with a subject and generate assessment data representing physiological parameters; and
a control unit operatively connected to the pulsed electromagnetic field module, the bioelectric stimulation module, and the analyzer, the control unit configured to coordinate a therapeutic protocol and generate a health assessment report based on the assessment data,
wherein the control unit is configured to modify at least one parameter of the current-limited stimulation signal based on at least one of (i) the assessment data or (ii) a monitored electrical characteristic associated with the practitioner-assisted conduction path.

2. The bioelectric therapy system of claim 1, wherein the pulsed electromagnetic field module comprises electromagnetic coil assemblies positioned within a subject positioning apparatus configured to deliver electromagnetic fields during treatment sessions.

3. The bioelectric therapy system of claim 1, wherein the bioelectric stimulation module comprises current limiting circuitry and an isolation barrier configured to limit output current and electrically isolate the stimulation signal from a mains power source.

4. The bioelectric therapy system of claim 1, wherein the practitioner-assisted conduction path includes: (i) at least one subject electrode pad coupled to a first electrode terminal, (ii) at least one practitioner return pad coupled to a second electrode terminal, and (iii) a conductive path through a practitioner when the practitioner contacts the practitioner return pad and touches the subject.

5. The bioelectric therapy system of claim 1, wherein the therapeutic protocol comprises a stimulation sequence across a plurality of anatomical zones including at least a spinal axis zone, an anterior upper extremities zone, a cervical/cranial/auricular zone, a posterior upper extremities zone, and a lower extremities and plantar surfaces zone.

6. The bioelectric therapy system of claim 4, further comprising contact detection circuitry configured to inhibit or terminate stimulation unless at least (i) subject contact at the subject electrode pad, (ii) practitioner contact at the practitioner return pad, and (iii) a conductive condition indicative of practitioner hand contact with the subject are detected.

7. A method for bioelectric therapy comprising:
acquiring baseline assessment data representing physiological parameters of a subject using an analyzer that measures at least one biophysical signal;
exposing the subject to a pulsed electromagnetic field generated by an electromagnetic field module for a predetermined duration;
configuring a practitioner-assisted conduction path by coupling at least one subject electrode pad to the subject and coupling at least one practitioner return pad to a practitioner support surface;
verifying a contact condition indicating subject contact, practitioner contact, and practitioner hand contact with the subject prior to enabling stimulation;
applying bioelectric stimulation by causing a practitioner to contact the practitioner return pad and contact the subject with the practitioner's hands such that a current-limited stimulation signal is delivered to targeted anatomical regions;
acquiring post-stimulation assessment data representing physiological parameters of the subject using the analyzer; and
generating a health assessment report based on the baseline assessment data and the post-stimulation assessment data.

8. The method of claim 7, wherein the predetermined duration is between about 2 minutes and about 20 minutes.

9. The method of claim 7, wherein the predetermined duration is about 8 minutes.

10. The method of claim 7, further comprising applying a topical preparation to at least one anatomical region prior to applying bioelectric stimulation.

11. The method of claim 7, wherein applying bioelectric stimulation comprises sequentially stimulating a plurality of zones targeting different anatomical regions in accordance with a stored protocol.

12. The method of claim 7, further comprising monitoring an electrical characteristic associated with the practitioner-assisted conduction path and adjusting a stimulation parameter based on the monitored electrical characteristic.

13. The method of claim 7, further comprising storing session results in a data store and generating a longitudinal trend display across a plurality of sessions.

14. An apparatus for bioelectric therapy comprising:
a bioelectric stimulation circuit configured to output a current-limited stimulation signal;
at least one subject electrode pad electrically coupled to a first output terminal of the bioelectric stimulation circuit;
at least one practitioner return pad electrically coupled to a second output terminal of the bioelectric stimulation circuit; and
contact detection circuitry configured to inhibit output of the current-limited stimulation signal unless at least (i) a subject contact at the subject electrode pad, (ii) a practitioner contact at the practitioner return pad, and (iii) a conductive condition indicative of practitioner hand contact with the subject are detected.

15. The apparatus of claim 14, wherein the bioelectric stimulation circuit is configured to output pulsed stimulation at a frequency between about 0.5 Hz and about 500 Hz.

16. The apparatus of claim 14, wherein the bioelectric stimulation circuit is configured to limit output voltage to less than about 30 volts peak and to limit output current to less than about 10 milliamps.

17. The apparatus of claim 14, further comprising contact monitoring circuitry configured to monitor a conductive condition associated with the practitioner-assisted conduction path and provide an operator indication based on changes in the conductive condition.

18. The apparatus of claim 14, further comprising a controller configured to terminate stimulation when contact is lost or when a monitored conductive condition falls outside a threshold range.

19. The apparatus of claim 14, further comprising a pulsed electromagnetic field module configured to perform a conditioning phase prior to stimulation.

20. The apparatus of claim 14, further comprising a data store configured to store health assessment reports and session metadata for longitudinal tracking.
